(19) United States
(12) Patent Application Publication
(10) Pub. No.: US 2019/0254312 A1
(43) Pub. Date: Aug. 22, 2019

(54) Vitamin D Compounds and Methods for Preparing Same

(71) Applicant: Vitamin Derivatives, Inc., Winterville, GA (US)

(72) Inventors: Hardy M. EDWARDS, III, Winterville, GA (US); George MAJETICH, Athens, GA (US); Richard HILL, Athens, GA (US); Dezhi SHA, Greenville, SC (US); George YIANNIKOUROS, Florence, SC (US)

(21) Appl. No.: 16/286,880

(22) Filed: Feb. 27, 2019

Related U.S. Application Data
(63) Continuation of application No. 15/171,133, filed on Jun. 2, 2016.
Application No. 15/171,133 is a continuation of application No. 13/381,671, filed on Apr. 11, 2012, now abandoned.
(60) Provisional application No. 61/222,470, filed on Jul. 1, 2009.

(51) Int. Cl. A23K 20/174; C30B 30/00; A61K 9/00; C07C 401/00; C30B 1/10; C30B 29/58; A61K 31/593; A23K 50/75

(52) U.S. Cl. CPC A23K 20/174; C30B 30/00; A61K 9/0056; C07C 401/00; C07B 2200/09; C30B 29/58; A61K 31/593; A23K 50/75; C07B 2200/13; C30B 1/10

(57) ABSTRACT

An air-stable, high-melt 1a-hydroxy-vitamin D₃ compound, methods for preparing an animal feed composition, methods of preparing 1a-hydroxy-vitamin D₃, methods of enhancing phytate phosphorus and calcium utilization, and an animal feed regime are provided.

### CROSS-REFERENCE TO RELATED APPLICATIONS

The present application claims priority to U.S. Provisional Application Ser. No. 61/222,470, filed Jul. 1, 2009, the contents of which are incorporated herein by reference in its entirety.

### FIELD OF THE INVENTION

This invention relates to the field of biochemistry, and particularly relates to animal feed compositions.

### BACKGROUND OF THE INVENTION

Animals, including humans, require phosphorus in their diets for proper growth and health. Farm animals are normally fed a grain-based animal feed. Most of these grain-based feeds contain from 50-80% of their total phosphorus as phytate phosphorus. Phytate phosphorus in plants occurs as the mixed calcium-magnesium-potassium salt of the organic compound, phytic acid.

Deficiencies in the animal's diet can lead to phosphorous deficiency (e.g., rickets) and bone health issues (e.g., tibial dyschondroplasia). Many animals are unable to utilize most of the phytate phosphorus they receive in their feed. Studies by Edwards and Veltmann, (J. Nutr. 113:1268-1575 (1983)) and Ballam et al., (Poultry Sci. 63:333-338 (1984)) with young broiler chickens fed corn-soybean diets indicate phytate phosphorus utilization of from only 10 to 53%. Feed consumed by these animals must be supplemented with inorganic phosphorus, such as in the form of dicalcium phosphate or defluorinated phosphate. The cost of phosphorus supplementation is high. In addition, the unused phytate phosphorus is excreted, creating phosphorus soil contamination and costly ecological problems.

The mechanisms involved in phytate phosphorus utilization by animals are unknown. Utilization of phytate phosphorus by chickens has been reviewed by several scientists including T. S. Nelson, (Poultry Sci. 46:862-871 (1967)). Phytate phosphorus utilization in broiler chickens has been shown by Edwards et al., (Poultry Sci. 67:1436-1446 (1988)) to be influenced by age. Other studies, have shown that phytate phosphorus utilization may be influenced by calcium, phosphorus, and aluminum levels in the diet (Lowe and Steenbock, Biochem J. 30:1991-1995 (1936), Common, Agric. Sci. 30:113-131 (1940), Edwards and Veltmann, J. Nutr. 113:1268-1575 (1983), Ballam et al., Poultry Sci. 63:333-338 (1984) and Sooncharernying and Edwards, Poultry Sci. 69(Suppl. 1):129 (1990)).

The active form of vitamin D₃, 1α,25-(OH)₂-vitamin D₃, regulates calcium and phosphorous homeostasis by enhancing their re-absorption by the proximal tubules of the kidneys and for bone mineralization in animals and man. In poultry, particularly, vitamin D₃ analogs can be up to thirty times more bioactive than vitamin D₂. Modern broiler chickens, for example, cannot make 1α,25-(OH)₂-vitamin D₃ fast enough to keep up with the physiological needs for calcium and phosphorous uptake.

The cost of producing the requisite large quantities of compositions containing vitamin D₃ analogs required to treat animals, however, poses numerous challenges. For example, due to instability and decomposition at ambient conditions, vitamin D₃ remains difficult to manufacture and store, especially in the quantities needed for mass administration to animals such as chickens (see methods set forth in “A Direct, Regio- and Stereoselective 1α-Hydroxylation of (5E)-Calciferol Derivatives”, Andrews, D. R., et al., J. Org. Chem., 1986, 51 (9), 1635-1637). Thus, there remains a need for high-melt, air-stable vitamin D₃ compositions as well as efficient methods of preparing such vitamin D₃ compositions in sufficiently large amounts for incorporation into various administration routes, including feed compositions. There further remains a need for methods that would increase phytate phosphorus utilization, increase dietary calcium utilization, optimize bone health and increase carcass quality and yields.

### SUMMARY OF THE INVENTION

According to one aspect, the present invention provides an air-stable, high-melt 1α-hydroxy-vitamin D₃ compound (alfacalcidol-1α,3β,5Z,7E-9,10-secocholesta-5,7,10(19)-triene-1,3-diol—illustrated below), crystalline hydrates, solvates, polymorphs and pharmaceutically acceptable salts thereof.

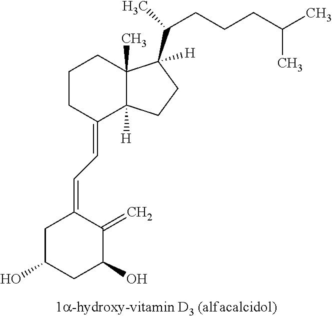 [SMILES: C=C1/C(=C\C=C2/CCC[C@]3(C)[C@@H]([C@H](C)CCCC(C)C)CC[C@@H]23)C[C@@H](O)C[C@@H]1O]

According to another aspect, the present invention provides a method of preparing a 1α-hydroxy-vitamin D₃ compound, crystalline hydrates, solvates, polymorphs and pharmaceutically acceptable salts thereof.

According to another aspect, the present invention provides methods of preparing an animal feed composition, including poultry feeds, comprising 1α-hydroxy-vitamin D₃ and crystalline hydrates, solvates, polymorphs and pharmaceutically acceptable salts thereof.

According to another aspect, the present invention provides methods of enhancing phytate phosphorus and calcium utilization by animals comprising the steps of administering to the animal an amount effective of 1α-hydroxy-vitamin D₃ in combination with an animal feed for growing animals. The animal feed comprises calcium or phosphorous or a combination thereof and the concentration of 1α-hydroxy-vitamin D₃ is at least 1 microgram of 1α-hydroxy-vitamin D₃ per kilogram of animal feed.

According to another aspect, the present invention provides an animal feed regime comprising 1α-hydroxy-vitamin D₃ or an animal feed composition comprising 1α-hydroxy-vitamin D₃ and instructions regarding effectively increasing phytate phosphorus utilization.

Combinations of aspects and embodiments form further embodiments of the present invention.

### BRIEF DESCRIPTION OF THE FIGURES

FIG. 1 illustrates the synthesis of a 1α-hydroxy-vitamin D₃ sample.

FIG. 2 is an x-ray powder diffraction pattern illustrating the observed peaks of a 1α-hydroxy-vitamin D₃ sample.

FIG. 3 is an indexed x-ray powder diffraction pattern illustrating the observed peaks of a 1α-hydroxy-vitamin D₃ sample.

FIG. 4 is a differential scanning calorimetry thermogram of a 1α-hydroxy-vitamin D₃ sample.

FIG. 5 is an infrared spectrum of a 1α-hydroxy-vitamin D₃ sample.

FIG. 6 is a thermogravimetric thermogram of a 1α-hydroxy-vitamin D₃ sample.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the embodiments.

### Methods of Preparing 1α-Hydroxy-Vitamin D₃

Efficient, scalable methods of preparing 1α,3β,5Z,7E-9,10-secocholesta-5,7,10(19)-triene-1,3-diol (1α-hydroxy-vitamin D₃) are provided. The crystalline hydrates, solvates, polymorphs and pharmaceutically acceptable salts thereof are may also be prepared. In one embodiment of the method of preparing the 1α-hydroxy-vitamin D₃ compound, vitamin D₃ may be treated with sulfur dioxide to produce two cyclic adducts which are protected via a silicon-protecting group. These protected adducts may undergo base-catalyzed sulfur dioxide removal and rearrangement to a single silicon-protected 5,6-trans-vitamin D₃. Allylic oxidation may then afford the corresponding 1α-hydroxy derivative, which is then de-protected to yield crystalline 1α-hydroxy-5,6-trans-vitamin D₃. Photochemical isomerization of the 1α-hydroxy-5,6-trans-vitamin D₃ may yield 1α-hydroxy-vitamin D₃ which undergoes final purification via polish filtration and direct crystallization.

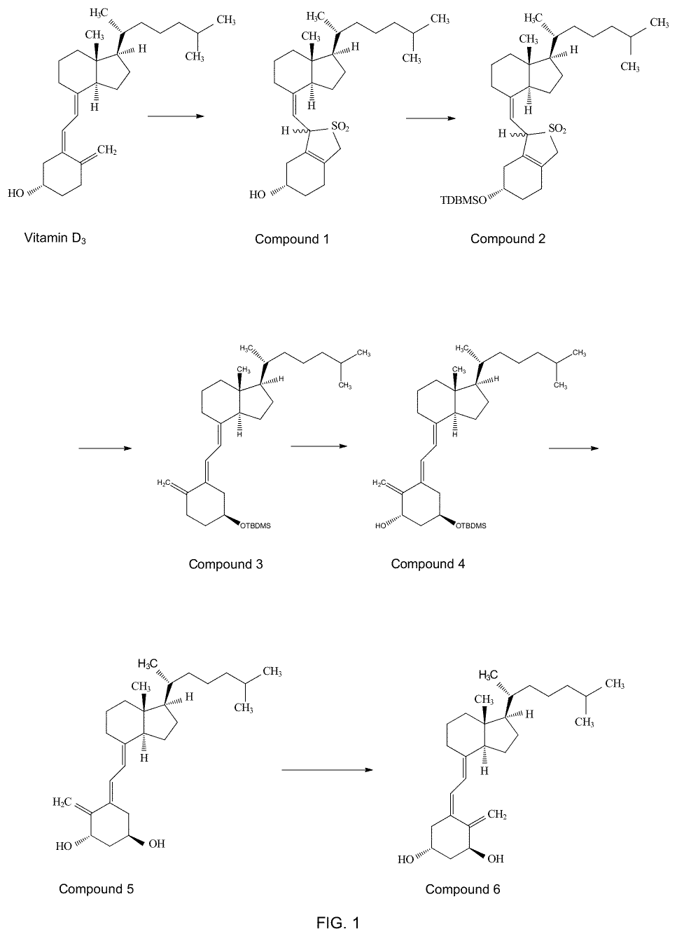

In one embodiment of the method, vitamin D₃ may be dissolved in three volumes of dichloromethane and the resulting solution cooled to approximately −15 to −20° C. Approximately 3.3 equivalents of sulfur dioxide gas may be charged while maintaining the temperature below −5° C. After the addition is complete, the reaction solution may be stirred for approximately one hour at the same temperature. The reaction progress may be monitored by silica gel thin-layer chromatograpy (TLC) [TLC In-Process Control (IPC): 0.25 mL reaction solution in 3 mL CH₂Cl₂; 60% EtOAc/Hexanes, phosphomolybdic acid (PMA) stain]. Once the reaction is complete, the solution may be warmed to approximately 10-15° C. while purging with nitrogen. The solvent may then be removed under vacuum thereby resulting in a viscous compound mixture (see Compound 1 of FIG. 1).

In one embodiment, the viscous compound mixture (e.g., Compound 1) may be dissolved in three volumes of dichloromethane. Approximately 1.2 equivalents of imidazole and 1.1 equivalents of t-butyldimethylsilyl chloride (TBDMSCl) may be charged into the dichloromethane solution. The reaction may be stirred at ambient temperature for approximately 2.5 to 4 hours. The reaction progress may be monitored by silica gel TLC [TLC IPC: 0.25 mL of reaction solution into 1 mL H₂O and 3 mL methyl t-butyl ether (MTBE); 60% EtOAc/Hexanes, PMA stain]. Additional imidazole and t-butyldimethylsilyl chloride may be added as needed to drive the reaction to completion. When the reaction is complete, the ammonium salts may be filtered off and the filter cake washed at least twice with one volume of dichloromethane. The solvent may be removed under vacuum resulting in a viscous silyl-protected liquid compound mixture (see Compound 2 of FIG. 1).

The viscous silyl-protected liquid compound mixture (e.g., Compound 2) may be dissolved in ten volumes of methyl t-butyl ether (MTBE). A solution of sodium hydroxide may be prepared by dissolving three equivalents of sodium hydroxide in five volumes of water and added to the reaction solution. The reaction mixture may be refluxed for approximately 24 to 30 hours to effect extrusion and removal of sulfur dioxide. When the reaction is complete, the reaction mixture may be cooled to ambient temperature, the two layers are separated, and the organic layer washed two times with four volumes of 5% brine solution. The solvent may be removed under vacuum. The crude product may be dissolved in 2.5 volumes of dichloromethane, and the solvent removed under vacuum. The resulting product may be once more dissolved in 2.5 volumes of dichloromethane with the solvent removed under vacuum thereby resulting in a viscous silyl-protected 5,6-trans-vitamin D₃ (see Compound 3 of FIG. 1).

The silyl-protected 5,6-trans-vitamin D₃ (e.g., Compound 3) may be combined with 1.3 equivalents of N-methylmorpholine N-oxide, 0.15 equivalents of N-methylmorpholine, and 0.4 equivalents of selenium dioxide. The reaction mixture may be refluxed for approximately 8 to 10 hours. The reaction may be cooled to ambient temperature. The solids may be filtered off through a Celite® pad and the solids washed with two volumes of dichloromethane. Any volatiles may be removed under vacuum. The resulting crude product may be dissolved in 2.5 volumes of tetrahydrofuran and the solvent removed under vacuum. The resulting product may be once more dissolved in 2.5 volumes of dichloromethane with the solvent removed under vacuum. The resulting crude product may be dissolved in ten volumes of tetrahydrofuran thereby producing a solution of dried 1α-hydroxy derivative (see Compound 4 of FIG. 1).

Approximately 1.5-1.8 equivalents of t-butylammonium fluoride may be charged to the 1α-hydroxy derivative (e.g., Compound 4) in the ten volumes of tetrahydrofuran and heated to 40° C. for approximately 24 to 30 hours to remove the silicon-based protecting group. When complete, the reaction may be cooled to ambient temperature and treated with one volume of saturated aqueous sodium bicarbonate solution. The layers may be separated and the organic layer may be concentrated under vacuum. The crude product may be dissolved in at least one organic solvent and concentrated under vacuum. In one embodiment, the crude product may be dissolved in two volumes of ethyl acetate. The solids residue may be combined with at least one organic solvent and water. In one embodiment, the solids residue may be combined with four volumes of ethyl acetate, six volumes of n-heptane, and seven volumes of water. The resulting solution may be stirred for approximately two hours to effect crystallization. The aqueous layer may be separated and the crystalline solids filtered and washed. In one embodiment, the filtered crystalline solids may be washed with two volumes of an organic solvent such as, for example, chloroform, 1-hexanol, isopropyl acetate, isobutyl acetate, isoamyl acetate, benzene, toluene, n-hexane, n-heptane, heptanes, or combinations thereof. In a preferred embodiment, the filtered crystalline solids may be washed with two volumes of n-heptane, heptanes, or a combination thereof. The solid 1α-hydroxy-5,6-trans-vitamin D₃ (see Compound 5 of FIG. 1) may be dried overnight. The combined yield of 1α-hydroxy-5,6-trans-vitamin D₃ from vitamin D₃ is approximately 30 to 40%. The typical purity of 1α-hydroxy-5,6-trans-vitamin D₃ is greater than 94.5%.

To obtain a purity of greater 94.5%, less pure batches of solid 1α-hydroxy-5,6-trans-vitamin D₃ may be dissolved in at least one water-miscible organic solvent, the solid impurities filtered, and the solids washed at least once with at least one water-miscible organic solvent. In one embodiment, solid 1α-hydroxy-5,6-trans-vitamin D₃ may be dissolved in five volumes of tetrahydrofuran, the solid impurities filtered, and the solids washed with two volumes of tetrahydrofuran. The solvent may be removed under vacuum. The partially purified 1α-hydroxy-5,6-trans-vitamin D₃ may then be treated with at least one organic solvent. In one embodiment, the partially purified 1α-hydroxy-5,6-trans-vitamin D₃ may be treated with 2.5 volumes of n-heptane, heptanes, or a combination thereof. The solvent may then be removed under vacuum and repeated, if necessary. The solid residue may be titurated with ten volumes of an organic solvent such as, for example, n-heptane, heptanes, or a combination thereof. The resulting 1α-hydroxy-5,6-trans-vitamin D₃ may be filtered, and washed with 1 to 2 volumes of an organic solvent such as, for example, n-heptane, heptanes, or a combination thereof. The purified 1α-hydroxy-5,6-trans-vitamin D₃ may then be air-dried overnight.

Approximately 1.0 kg of purified 1α-hydroxy-5,6-trans-vitamin D₃ may be dissolved in at least one water-miscible organic solvent. In one embodiment, the purified 1α-hydroxy-5,6-trans-vitamin D₃ may be first dissolved in tetrahydrofuran. In a preferred embodiment, the purified 1α-hydroxy-5,6-trans-vitamin D₃ may be first dissolved ten to twelve volumes of tetrahydrofuran. The resulting solution may be polish filtered to remove any particulate matter. The solution may be circulated through a mercury low pressure or medium pressure UV photochemical reactor for at least 15 hours with or without trace quantities of photochemical sensitizers such as, for example, acridine or Rose Bengal. The progress of the photochemical cis-trans isomerization may be monitored by HPLC. When the photoreaction is complete, the 1α-hydroxy-vitamin D₃ containing solution may be concentrated under vacuum.

In a preferred embodiment, the resulting solution of 1α-hydroxy-vitamin D₃ (see Compound 6 in FIG. 1) in tetrahydrofuran may undergo a solvent exchange (e.g., solvent switch). In one embodiment, the solvent exchange is carried out under vacuum. In one embodiment, the tetrahydrofuran is exchanged with a second organic solvent. In a preferred embodiment, the second organic solvent is n-heptane, heptanes, or a combination thereof. In a particularly preferred embodiment, the solvent exchange may be carried out at least four times with 1.5 volumes of n-heptane under vacuum. The solution may be concentrated under vacuum to approximately two volumes of the second organic solvent during the last exchange. The solution may be allowed to crystallize for at least twelve hours. The product may be filtered and dried to a loss-on-drying (LOD) of less than 2%. In a preferred embodiment, the recovered 1α-hydroxy-vitamin D₃ is substantially free of tetrahydrofuran (<1% of tetrahydrofuran co-solvent).

In one embodiment, the yield of 1α-hydroxy-vitamin D₃ from 1α-hydroxy-5,6-trans-vitamin D₃ is at least 70%. In a preferred embodiment, the yield of 1α-hydroxy-vitamin D₃ is at least 75%. In one embodiment, the 1α-hydroxy-vitamin D₃ purity is at least 90%. In a preferred embodiment, the 1α-hydroxy-vitamin D₃ purity is at least 96%.

In one embodiment, the method of preparing 1α-hydroxy-vitamin D₃ may be scaled up to produce kilogram commercial quantities. In one embodiment, the method of preparing 1α-hydroxy-vitamin D₃ does not require the use of column chromatography.

In one embodiment, 1α-hydroxy-5,6-trans-vitamin D₃ and the resulting 1α-hydroxy-vitamin D₃ are crystalline solids that do not significantly decompose on air drying at ambient temperature. In one embodiment, the 1α-hydroxy-5,6-trans-vitamin D₃ and the resulting 1α-hydroxy-vitamin D₃ are highly crystalline stable solids contrary to conventional vitamin D₂ and D₃ derivatives which are unstable in air leading to isomerization, oxidization and decomposition.

In one embodiment, the methods provide a 1α-hydroxy-vitamin D₃ compound that has a melting point of about 140° C. to about 144° C. In another embodiment, the methods provide a 1α-hydroxy-vitamin D₃ compound that melts at a temperature of at least 140° C. In a preferred embodiment, the 1α-hydroxy-vitamin D₃ compound melts at a temperature of at least 142° C. In a particularly preferred embodiment, the 1α-hydroxy-vitamin D₃ compound melts at a temperature of at least 143° C.

### Methods of Preparing Dried 1α-Hydroxy-Vitamin D₃ Formulation

In one embodiment, the methods provided herein may include the step of drum-drying the 1α-hydroxy-vitamin D₃ obtained from the methods described herein. In one embodiment, the step of drum-drying the 1α-hydroxy-vitamin D₃ is conducted via a batch process. In one embodiment, the step of drum-drying includes the step of adding starch, at least one vegetable oil, at least one emulsifier, at least one food dye, and at least one preservative or antioxidant, alone or in various combinations with the 1α-hydroxy-vitamin D₃.

In one embodiment, a source of starch such as, for example, NADEX® 772 (available from National Starch Food Innovation, Bridgewater, N.J.), may be used as a bulk carrier. In one embodiment, maltodextrin (available from Tate & Lyle, London, United Kingdom), Bridgewater, N.J.), enzymatically derived from starch, may be used alone or in combination with another starch source as a bulk carrier. In one embodiment, starch is added in an amount between 87% and 97%. In a preferred embodiment, starch is added in an amount between 89% and 95%. In a particularly preferred embodiment, starch is added in an amount of about 92%.

In one embodiment, at least one vegetable oil is added to aid in the dissolving and stabilizing of various components of the 1α-hydroxy-vitamin D₃ drum-dried formulation. In one embodiment, the at least one vegetable oil is added in an amount between 1.00% and 12.00%. In a preferred embodiment, the at least one vegetable oil is added in an amount between 5.00% and 7.00%. In a particularly preferred embodiment, the at least one vegetable oil is added in an amount of about 6.00%. In a preferred embodiment, the vegetable oil is olive oil, palm oil, soybean oil, canola oil, pumpkin seed oil, corn oil, sunflower oil, safflower oil, peanut oil, grape seed oil, sesame oil, argan oil, rice oil, or a combination thereof. In a particularly preferred embodiment, the vegetable oil is peanut oil.

In one embodiment, the at least one emulsifier is added in an amount between 0.4% and 1.6%. In a preferred embodiment, the at least one emulsifier is added in an amount between 0.7% and 1.3%. In a particularly preferred embodiment, the at least one emulsifier is added in an amount of about 1.00%. In a preferred embodiment, the at least one emulsifier is glyceryl monostearate, polyethylene glycol monolaurate, calcium stearoyl lactate, sodium stearoyl lactate, sorbitan monostearate, polyoxyethylene sorbitan monostearate, sucrose monopalmitate and sucrose monostearate or combinations thereof. In a particularly preferred embodiment, the at least one emulsifier is sorbitan monostearate.

In one embodiment, the at least one food dye is added in an amount between 0.10% and 0.40%. In a preferred embodiment, the at least one food dye is added in an amount between 0.20% and 0.30%. In a particularly preferred embodiment, the at least one food dye is added in an amount of about 0.25%. In a preferred embodiment, the food dye is FD&C Green #3, although any triarylmethane food dye may be added to achieve a desired color.

In one embodiment, the at least one preservative or antioxidant is added in an amount between 0.10% and 0.30%. In a preferred embodiment, the at least one preservative or antioxidant is added in an amount between 0.15% and 0.25%. In a particularly preferred embodiment, the at least one preservative or antioxidant is added in an amount of about 0.20%. In a preferred embodiment, the at least one preservative or antioxidant is butylated hydroxyanisole, butylated hydroxytoluene, propyl gallate, D-sodium isoascorbate, polyphenol, vitamin E, or combinations thereof. In a particularly preferred embodiment, the preservative or antioxidant is butylated hydroxytoluene (BHT).

In one embodiment, a second preservative is added in an amount between 0.10% and 0.30%. In a preferred embodiment, the second preservative is added in an amount between 0.15% and 0.25%. In a particularly preferred embodiment, the second preservative is added in an amount of about 0.20%. In a preferred embodiment, the second preservative is sorbic acid, benzoic acid, propionic acid, or combinations thereof. In a particularly preferred embodiment, the second preservative is sorbic acid.

In one embodiment, a third preservative is added in an amount between 0.04% and 0.16%. In a preferred embodiment, the third preservative is added in an amount between 0.07% and 0.13%. In a particularly preferred embodiment, the third preservative is added in an amount of about 0.10%. In a preferred embodiment, the third preservative is sodium benzoate, potassium sorbate, adenosine monophosphate, or combinations thereof. In a particularly preferred embodiment, the third preservative is sodium benzoate.

In one embodiment, the 1α-hydroxy-vitamin D₃ is added in an amount between 0.01% and 0.07%. In a preferred embodiment, the 1α-hydroxy-vitamin D₃ is added in an amount between 0.03% and 0.05%. In a particularly preferred embodiment, the 1α-hydroxy-vitamin D₃ is added in an amount of about 0.04%.

A particularly preferred embodiment of a drum-dried 1α-hydroxy-vitamin D₃ formulation is set forth in Table I.

TABLE 1

| Ingredient | % |
|---|---|
| Starch | 92.21 |
| Peanut Oil | 6.00 |
| Sorbitan Monostearate | 1.00 |
| FD&C Green #3 | 0.25 |
| Sorbic Acid | 0.20 |
| BHT | 0.20 |
| Sodium Benzoate | 0.10 |
| 1α-hydroxy-vitamin D₃ | 0.04 |
| Total | 100.00 |

In one embodiment, batching may be performed separately for a water phase and an oil phase. In a preferred embodiment, the water phase, starch, sorbic acid, dye and sodium benzoate may be added to 60° C. water and stirred vigorously until all components are dissolved. Separately, a vegetable oil such as, for example, peanut oil, may be heated to approximately 58° C. to 60° C. after which sorbitan monostearate, BHT and 1α-hydroxy-vitamin D₃ may be added. The solution may be stirred vigorously until all components are dissolved.

In one embodiment, the water phase and oil phase solutions may be combined and mixed thoroughly. Upon combining the water phase and oil phase, a total solids content of about 20% to 60% is obtained in the final mixture. In a preferred embodiment, the resulting solids content is about 30% to about 50%. In a particularly preferred embodiment, the resulting solids content is about 40%.

This combined emulsified mixture, still at 60° C., may be introduced to at least one drum-drier. The drum-drier rolls may be maintained at a temperature of 185° C. The resulting drum-dried 1α-hydroxy-vitamin D₃ formulation may be peeled off the rolls, run through a mill and screened to where greater than 98% of the drum-dried 1α-hydroxy-vitamin D₃ formulation flows through a 20 mesh screen and less than 30% of the drum-dried 1α-hydroxy-vitamin D₃ formulation flows through a 60 mesh screen.

In an alternative embodiment, the combined emulsified mixture may be introduced to at least one spray-drier. The emulsified mixture may be heated up to about 200° C. for up to about one second in the spray-drier. In a preferred embodiment, the resulting spray-dried 1α-hydroxy-vitamin D₃ formulation that flows from the spray-drier is ready for packaging.

In one embodiment, 1α-hydroxy-vitamin D₃ may be added to a final animal feed. In a preferred embodiment, the drum-dried or spray-dried 1α-hydroxy-vitamin D₃ may be added to a commercial vitamin pre-mix which is then added to a finished animal feed. In another embodiment, the 1α-hydroxy-vitamin D₃ may be added to an animal feed during manufacture of the animal feed. In a preferred embodiment, drum-dried or spray-dried 1α-hydroxy-vitamin D₃ is added during manufacture of the animal feed.

### Premix Formulation and Animal Feed Composition Preparation

In one embodiment, a carrier including solid components such as, for example, standard ground limestone and ground rice hull and a liquid component such as, for example, mineral oil, may be mixed or blended with 1α-hydroxy-vitamin D₃ to form an animal feed premixture composition. In one embodiment, at least about 15 grams of 1α-hydroxy-vitamin D₃ may be blended with each pound of carrier to form an animal feed premixture composition. In one embodiment, at least about 20 grams of 1α-hydroxy-vitamin D₃ may be blended with each pound of the carrier to form an animal feed premixture composition. In a particularly preferred embodiment, at least about 22.68 grams of 1α-hydroxy-vitamin D₃ may be blended with each pound of carrier to form the animal feed premixture composition.

In one embodiment, the animal feed premixture composition may be added to a finished animal feed. In an alternative embodiment, the animal feed premixture composition including drum-dried 1α-hydroxy-vitamin D₃ may be added to a finished animal feed. In either embodiment, the animal feed premixture composition may be added to a finished animal feed at a rate of at least about one-fourth pound (¼ lb) per ton of finished feed. In a preferred embodiment, the animal feed premixture composition may be added to finished animal feed at a rate of at least about one-third pound (⅓ lb) per ton of finished feed. In a particularly preferred embodiment, the animal feed premixture composition may be added to finished animal feed at a rate of at least about one-half pound (½ lb) per ton of the finished feed to arrive at a dosing of at least about 5 μg of 1α-hydroxy-vitamin D₃/kg of finished feed which is of equivalent potency to finished feeds containing 40-50 μg/kg of vitamin D₃.

In one embodiment, the carrier may be used to convey 1α-hydroxy-vitamin D₃ into a feed mixer from an overhead micro-bin. The ratio of ground limestone (16×120) to ground rice hulls (80×30) is 60:40 on a weight basis. In one embodiment, at least about 0.1% mineral oil is incorporated to reduce dust in the resulting mixture. In preferred embodiment, at least about 0.5% mineral oil is incorporated. In a particularly preferred embodiment, at least about 1% mineral oil is incorporated.

In one embodiment, the animal feed to which the 1α-hydroxy-vitamin D₃ may be added may contain trace minerals, such as, for example iron, calcium (e.g., oyster shells or limestone), copper, manganese, zinc, iodine, selenium, sodium selenite, feed additives, or a combination thereof. The animal feed may also include base vitamins or nutrients such as, for example, dicalcium phosphate, vitamin A, E, D, B₁₂, riboflavin, pantothenic acid, niacin, biotin, or combinations thereof. The animal feed may also contain at least one protein or grain co-product and at least one fat or carbohydrate source to meet the specific animal's dietary needs such as, for example, poultry fat, soybean meal, corn, maize, wheat, oats, milo, barley, peanut meal, cottonseed meal, fish byproducts, blood meal or a combination thereof. In one embodiment, additional trace minerals, base vitamins or nutrients, one protein or grain co-product, fat or carbohydrates sources may be added to the animal feed composition.

In one embodiment, the animal feed or animal feed premixture composition can additionally include phytase to enhance phytate phosphorous utilization to an even greater extent compared to conventional animal feed. Phytase is an enzyme that converts phytate phosphorous to inorganic phosphate ions. In a preferred embodiment, phytase may be added to the animal feed or animal feed premixture composition by adding the phytase to the feed as recommended by the various manufacturers of phytase. In one embodiment, the resulting animal feed contains from about 10 to about 30,000 units of active phytase per kilogram of feed. In a preferred embodiment, the resulting animal feed contains from about 20 to about 20,000 units of active phytase per kilogram of feed. In a particularly preferred embodiment, the resulting animal feed contains from about 30 to about 30,000 units of active phytase per kilogram of feed.

In an alternative embodiment, 1α-hydroxy-vitamin D₃ may be added to a vitamin premix manufactured by a third party. In yet another alternative embodiment, drum-dried 1α-hydroxy-vitamin D₃ may be added to a vitamin premix manufactured by a third party. In either embodiment, the 1α-hydroxy-vitamin D₃ may be added to a vitamin premix a rate of at least about 1 gram of 1α-hydroxy-vitamin D₃ per finished ton of feed. In a preferred embodiment, 1α-hydroxy-vitamin D₃ is added to an a vitamin premix manufactured by a third party at a rate of at least about 8 grams of 1α-hydroxy-vitamin D₃ per finished ton of feed. In a particularly preferred embodiment, 1α-hydroxy-vitamin D₃ is added to a vitamin premix manufactured by a third party such that the inclusion rate of the 1α-hydroxy-vitamin D₃ via the third party vitamin premix into the finished feed is 12.5 grams per metric ton.

In one embodiment, the animal feed may be a chicken feed to which the 1α-hydroxy-vitamin D₃ mixture may be added in the ranges of amounts set forth in Table 2. The amounts indicated are given in percent by weight. It will be understood by those skilled in the art that the 1α-hydroxy-vitamin D₃ mixture described herein can also be fed in combination with other commercially formulated or similar feeds for chickens and other animals.

TABLE 2

| Ingredient | % |
|---|---|
| Ground Yellow Corn | 55.0-58.0 |
| Soybean meal (dehulled) | 34.0-36.0 |
| Poultry Fat (stabilized) | 4.0-6.0 |
| Iodized sodium chloride | 0.35-0.55 |
| D.L.-Methionine (98%) | 0.10-0.30 |
| 1α-hydroxy-vitamin D₃ mixture | 0.15-0.35 |
| Trace minerals | 0.05-0.15 |
| Se concentrate (0.02% from sodium | 0.04-0.06 |
| selenite) | |
| Dicalcium phosphate (feed grade) | 0.0-4.0 |
| Limestone | 0.1-0.3 |

### Methods of Use and Dosage

In one embodiment, 1α-hydroxy-vitamin D₃ may be administered to an animal in an optional pharmaceutically acceptable carrier. In one embodiment, 1α-hydroxy-vitamin D₃ may be administered via mixing with an appropriate animal feed. In one embodiment, the 1α-hydroxy-vitamin D₃ or the resulting animal feed provides a dietary source of vitamin D to an animal including, but is not limited to, swine, dogs, rabbits, cattle, fish, and fowl, such as, for example, cornish game hens, broilers, broiler-breeders, layers, pheasants, ducks and turkeys. In one embodiment, 1α-hydroxy-vitamin D₃ may be administered to animals via a water supply, by time or slow-release bolus or other controlled drug delivery device, an orally administered capsule, or by an injection orally, subcutaneously, intramuscularly, intravenously or intraperitoneally.

According to one embodiment, 1α-hydroxy-vitamin D₃ is administered to an animal which delivers a dosage at a level of at least 1 μg of 1α-hydroxy-vitamin D₃/kg of finished feed. According to a preferred embodiment, 1α-hydroxy-vitamin D₃ is administered to an animal which delivers a dosage at a level of at least 5 μg of 1α-hydroxy-vitamin D₃/kg of finished feed. In one embodiment, the vitamin D composition administration may be initiated at birth and continued throughout the animal's rapid growth stage. The amount of compound in the feed may be decreased over time to take into account the increased feed intake of the animal as it grows thereby allowing the producer to use cheaper feed for the older animal (e.g., fowl). The resulting 1α-hydroxy-vitamin D₃ enriched, finished feed provides a dietary source of vitamin D.

An animal feed regime is also provided. In one embodiment, the animal feed regime comprises 1α-hydroxy-vitamin D₃ or an animal feed composition comprising 1α-hydroxy-vitamin D₃ and instructions regarding effectively increasing phytate phosphorus utilization. While not intending to be bound by a particular theory, the inventors believe that when 1α-hydroxy-vitamin D₃ is fed, the 1α-hydroxy-vitamin D₃ is quickly hydroxylated to an active 1,25-(OH)₂-D₃. Calcium absorption, and as a result, phosphorus and the divalent trace mineral absorptions are also increased. With less calcium in the digestive tract to form insoluble fatty acid soaps, fat absorption is improved. Thus, an increase in phytate phosphorus utilization may result optimized bone health, an increased carcass quality, an increased carcass yield, and combinations thereof. Specifically, incidences of bone abnormalities, calcium and phosphorus-deficiency rickets and tibial dyschondroplasia can be lowered or completely eliminated by adding 1α-hydroxy-vitamin D₃ to animal diets. The 1α-hydroxy-vitamin D₃ is believed to work independently of exogenous phytase enzymes. Phytase works in the upper gastro-intestinal tract at low pH to aid in the digestion of phytate to phosphate and inositol. Activated 1α-hydroxy-vitamin D₃ works in the lower gastro-intestinal tract at high pH to aid in calcium, phosphorus and trace mineral absorptions. Although 1α-hydroxy-vitamin D₃ and phytase work together to increase phosphorus utilization, only 1α-hydroxy-vitamin D₃ targets bone tissue thereby reducing bone abnormalities.

Specific pharmacological responses observed may vary according to and depending on whether there are present pharmaceutical carriers, as well as the type of formulation and mode of administration employed, and such expected variations or differences in the results are contemplated in accordance with practice of the present invention.

### EXAMPLES

### Example 1

Experiments were conducted to determine the x-ray powder diffraction pattern of a 1α-hydroxy-vitamin D₃ sample. The x-ray powder diffraction pattern was collected with a PANalytical X'Pert PRO MPD diffractometer (available from PANalytical of Almelo, The Netherlands) using an incident beam of Cu radiation produced using an Optix long, fine-focus source. An elliptically graded multilayer mirror was used to focus Cu Kα x-rays through the sample and onto a detector. Prior to the analysis, a silicon standard specimen (NIST SRM 640c) was analyzed to verify the Si (111) peak position. A specimen of the sample was placed between 3-jim-thick films and analyzed in transmission geometry. A beam-stop was used to minimize the background generated by air. Soller slits for the incident and diffracted beams were used to minimize broadening from axial divergence. The diffraction pattern was collected using a scanning position-sensitive detector (X'Celerator available from PANalytical of Almelo, The Netherlands) located 240 millimeters from the specimen and processed with Data Collector software v. 2.2b.

Rounding algorithms were used to round each peak to the nearest 0.1° or 0.01°, depending upon the instrument used to collect the data and/or the inherent peak resolution. The locations of the peaks along the x-axis (° 20) were automatically determined using proprietary PatternMatch™ 3.0.1 software and rounded to one or two significant figures after the decimal point. Peak position variabilities were given to within ±0.1° 20 (see United States Pharmacopeia, USP 32, NF 27, Vol. 1, pg. 392, 2009). For d-space listings, the wavelength used to calculate d-spacings was 1.541874 A (a weighted average of the Cu—Kₐ1 and Cu—Kₐ2 wavelengths). Variability associated with d-spacing estimates was calculated from the United States Pharmacopeia recommendation at each d-spacing.

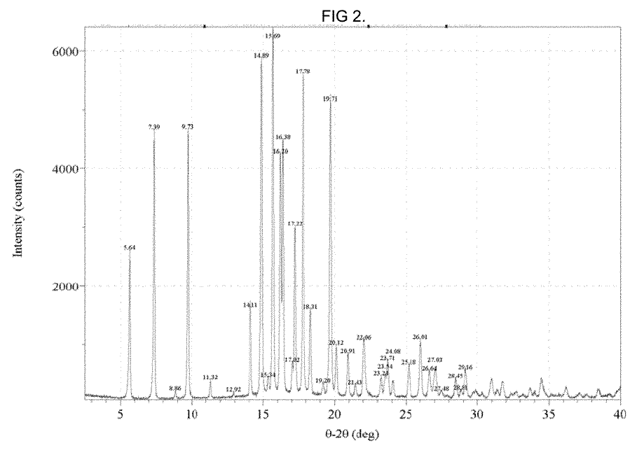

One x-ray powder diffraction pattern was analyzed. The x-ray powder diffraction pattern of 1α-hydroxy-vitamin D₃ exhibited relatively sharp peaks, indicating the sample was composed of crystalline material. Two lists of x-ray powder diffraction peak positions were made for the x-ray powder diffraction pattern of 1α-hydroxy-vitamin D₃. Observed peaks are shown in FIG. 2 and listed on Table 3. Prominent peaks, listed in Table 4, were selected from observed peaks by identifying preferably non-overlapping, low-angle peaks, with strong intensity.

TABLE 3

| °2θ | d space (Å) | Intensity (%) |
|---|---|---|
| 5.64 ± 0.10 | 15.661 ± 0.282 | 41 |
| 7.39 ± 0.10 | 11.963 ± 0.164 | 73 |
| 8.86 ± 0.10 | 9.981 ± 0.114 | 3 |
| 9.73 ± 0.10 | 9.091 ± 0.094 | 73 |
| 11.32 ± 0.10 | 7.819 ± 0.069 | 6 |
| 12.92 ± 0.10 | 6.852 ± 0.053 | 3 |
| 14.11 ± 0.10 | 6.278 ± 0.045 | 25 |
| 14.89 ± 0.10 | 5.949 ± 0.040 | 92 |
| 15.34 ± 0.10 | 5.775 ± 0.038 | 7 |
| 15.69 ± 0.10 | 5.649 ± 0.036 | 100 |
| 16.20 ± 0.10 | 5.473 ± 0.034 | 66 |
| 16.38 ± 0.10 | 5.412 ± 0.033 | 70 |
| 17.02 ± 0.10 | 5.211 ± 0.031 | 11 |
| 17.22 ± 0.10 | 5.151 ± 0.030 | 47 |
| 17.78 ± 0.10 | 4.988 ± 0.028 | 88 |
| 18.31 ± 0.10 | 4.845 ± 0.026 | 25 |
| 19.20 ± 0.10 | 4.624 ± 0.024 | 5 |
| 19.71 ± 0.10 | 4.505 ± 0.023 | 80 |
| 20.12 ± 0.10 | 4.414 ± 0.022 | 15 |
| 20.91 ± 0.10 | 4.249 ± 0.020 | 13 |
| 21.43 ± 0.10 | 4.147 ± 0.019 | 5 |
| 22.06 ± 0.10 | 4.029 ± 0.018 | 17 |
| 23.24 ± 0.10 | 3.827 ± 0.016 | 8 |
| 23.54 ± 0.10 | 3.779 ± 0.016 | 7 |
| 23.71 ± 0.10 | 3.753 ± 0.016 | 11 |
| 24.08 ± 0.10 | 3.697 ± 0.015 | 6 |
| 25.18 ± 0.10 | 3.537 ± 0.014 | 10 |
| 26.01 ± 0.10 | 3.425 ± 0.013 | 17 |
| 26.64 ± 0.10 | 3.346 ± 0.012 | 9 |
| 27.03 ± 0.10 | 3.298 ± 0.012 | 9 |
| 27.48 ± 0.10 | 3.246 ± 0.012 | 3 |
| 28.45 ±0.10 | 3.137 ± 0.011 | 7 |
| 28.81 ±0.10 | 3.099 ± 0.011 | 4 |
| 29.16 ± 0.10 | 3.063 ± 0.010 | 9 |

TABLE 4

| °2θ | d space (Å) | Intensity (%) |
|---|---|---|
| 5.64 ± 0.10 | 15.661 ± 0.282 | 41 |
| 7.39 ± 0.10 | 11.963 ± 0.164 | 73 |
| 9.73 ± 0.10 | 9.091 ± 0.094 | 73 |
| 14.11 ± 0.10 | 6.278 ± 0.045 | 25 |
| 14.89 ± 0.10 | 5.949 ± 0.040 | 92 |
| 15.69 ± 0.10 | 5.649 ± 0.036 | 100 |
| 16.20 ± 0.10 | 5.473 ± 0.034 | 66 |
| 16.38 ± 0.10 | 5.412 ± 0.033 | 70 |
| 17.21 ± 0.10 | 5.153 ± 0.030 | 46 |
| 17.78 ± 0.10 | 4.988 ± 0.028 | 88 |
| 18.31 ± 0.10 | 4.845 ± 0.026 | 25 |
| 19.71 ± 0.10 | 4.505 ± 0.023 | 80 |

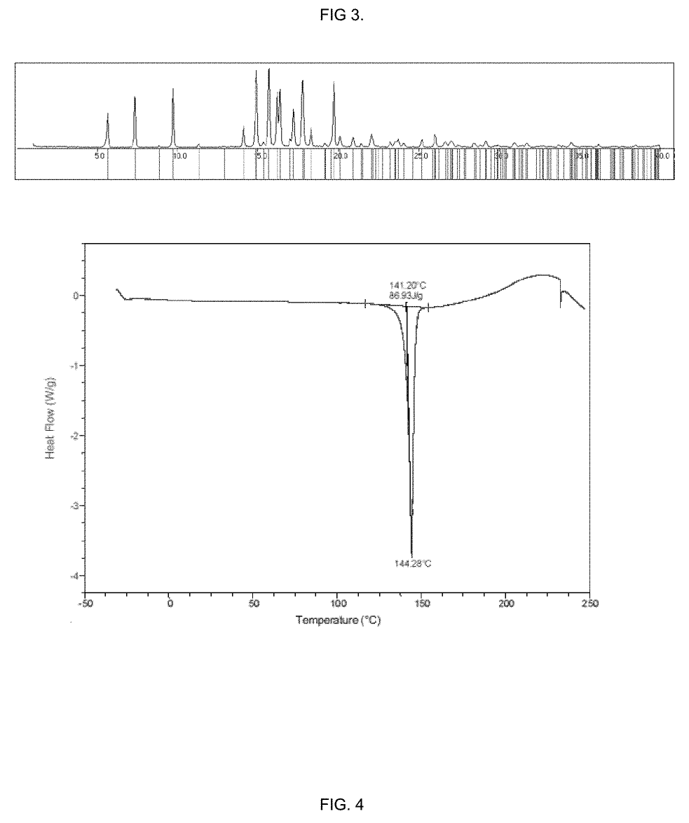

The indexed x-ray powder diffraction pattern of 1α-hydroxy-vitamin D₃ is illustrated in FIG. 3. Agreement between the allowed peak positions, marked with vertical bars in FIG. 3, and the observed peaks indicates a consistent unit cell determination. Successful indexing of the pattern indicates that the sample is composed primarily of a single crystalline phase. Space groups consistent with the assigned extinction symbol, unit cell parameters, and derived quantities are tabulated in Table 5.

TABLE 5

| | |
|---|---|
| Form/Pattern | 1 α-hydroxy-vitamin D₃ |
| Family and | Monoclinic |
| Space Group | P2₁ (#4) |
| Z′/Z | 1/2 |
| a (Å) | 12.001 |
| b (Å) | 6.846 |
| c (Å) | 15.694 |
| α (deg) | 90 |
| β (deg) | 95.52 |
| γ (deg) | 90 |
| Volume (Å³/cell) | 1283.4 |
| V/Z (Å³/asym. unit) | 641.7 |
| Assumed Composition a | C₂₇H₄₄O₂ |
| Density (g/cm³)^{a} | 1.037 |
| Weight Fraction Solvent (%)^{a} | N/A |

### Example 2

Differential scanning calorimetry analyses were performed using a TA Instruments 2920 differential scanning calorimeter (available from TA Instruments of New Castle, Del.). The sample was placed into an aluminum differential scanning calorimetry pan, and the weight was accurately recorded. The sample pan was covered with a lid and then crimped. The sample cell was equilibrated at −30° C. and heated under a nitrogen purge at a rate of 10° C./min, up to a final temperature of 250° C. Indium metal was used as the calibration standard.

The differential scanning calorimetry thermogram of the 1α-hydroxy-vitamin D₃ sample exhibited a sharp endotherm with the peak maximum at approximately 144° C. (onset: 141° C.)(See FIG. 4).

### Example 3

An infrared spectrum was acquired on a Magna-IR 860® Fourier transform infrared (FT-IR) spectrophotometer equipped with an Ever-Glo mid/far infrared source (spectrophotometer and IR source available from Thermo Fischer Scientific of Waltham, Mass.), an extended range potassium bromide (KBr) beamsplitter, and a deuterated triglycine sulfate (DTGS) detector. Wavelength verification was performed using standardized (NIST SRM 1921b) polystyrene film. An attenuated total reflectance (ATR) accessory (Thunderdome™, available from Thermo Spectra-Tech), with a germanium (Ge) crystal was used for data acquisition. Data was acquired from 4000 to 675 cm⁻¹ with an absolute threshold of 0.0028 and a sensitivity of 70. A background data set was acquired with a clean germanium crystal. A Log 1/R (R=reflectance) spectrum was obtained by taking a ratio of the two data sets against each other.

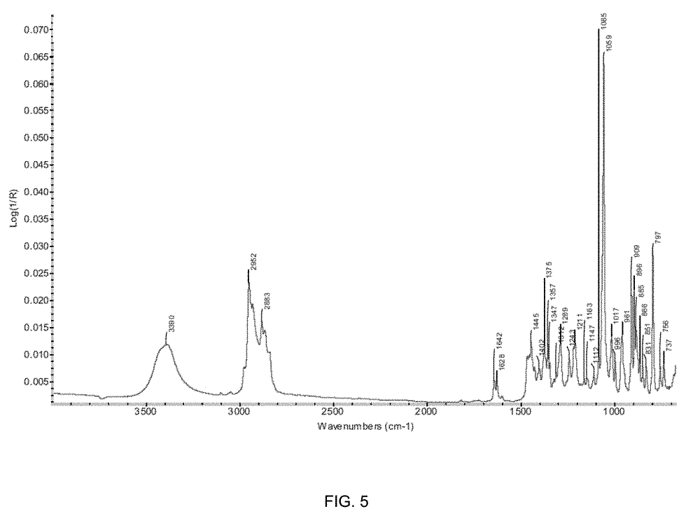

The IR spectrum of the 1α-hydroxy-vitamin D₃ sample displayed a flat baseline with well resolved and sharp bands. The peak lists generated are listed in Table 6 and illustrated in FIG. 5.

TABLE 6

| Position (cm⁻¹) | Intensity (Log (1/R)) |
|---|---|
| 737 | 0.0082 |
| 756 | 0.0076 |
| 797 | 0.0281 |
| 831 | 0.0075 |
| 851 | 0.0075 |
| 866 | 0.0081 |
| 885 | 0.0143 |
| 896 | 0.0226 |
| 909 | 0.0215 |
| 961 | 0.0135 |
| 996 | 0.0086 |
| 1017 | 0.0131 |
| 1059 | 0.0634 |
| 1085 | 0.0095 |
| 1112 | 0.0058 |
| 1147 | 0.0053 |
| 1163 | 0.0053 |
| 1211 | 0.012 |
| 1243 | 0.0089 |
| 1289 | 0.0119 |
| 1312 | 0.0066 |
| 1347 | 0.0083 |
| 1357 | 0.0118 |
| 1375 | 0.0115 |
| 1402 | 0.0072 |
| 1445 | 0.0121 |
| 1628 | 0.0044 |
| 1642 | 0.005 |
| 2883 | 0.0159 |
| 2952 | 0.0232 |
| 3390 | 0.0116 |

### Example 4

Thermogravimetric analysis of a 1α-hydroxy-vitamin D₃ sample was performed using a TA Instruments 2950 thermogravimetric analyzer (available from TA Instruments of New Castle, Del.). The sample was placed in an aluminum sample pan and inserted into the thermogravimetric furnace. The furnace was heated under nitrogen at a rate of 10° C./min, from room temperature to a final temperature of 350° C. Nickel and Alumel™ (available from Hoskins Manufacturing Company, Novi, Mich.) were used as calibration standards.

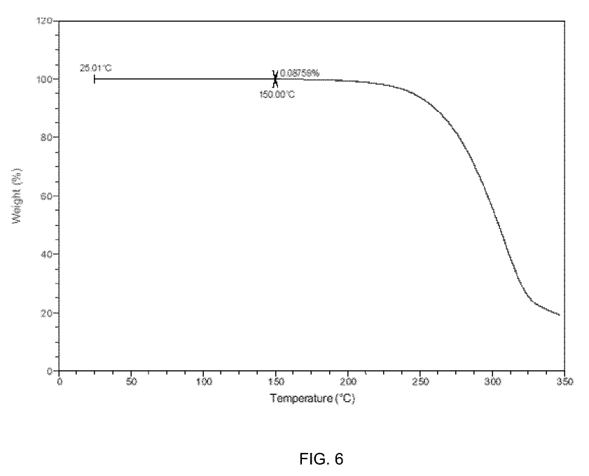

The thermogravimetric thermogram exhibited a minor weight loss of 0.09% from 25° C. to 150° C., which indicated the material was not a solvate or hydrate and decomposed above approximately 200° C. (See FIG. 6).

### Example 5

An optical rotation analysis of two 1α-hydroxy-vitamin D₃ samples was performed using AutoPol V polarimeter (available from Rudolph Research Analytical of Flanders, N.J.). The specific rotation of the 1α-hydroxy-vitamin D₃ samples was evaluated according to USP 781. Specifically, the specific rotation was determined by taking a 0.25 gram sample of 1α-hydroxy-vitamin D₃ and diluting to 50 mL with diethyl ether. The temperature was maintained at 25° C. The results are summarized in Table 7.

TABLE 7

| Sample No. | [a]²⁵ _{D} |
|---|---|
| 1 | +32.3 degrees (c = 0.50, ether) |
| 2 | +31.9 degrees (c = 0.50, ether) |

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1-29. (canceled)

30. A method for preparing 1α-hydroxy-vitamin D₃ comprising the steps of
treating vitamin D3 with sulfur dioxide to produce two cyclic compounds, each protected via a silicon-protecting group;
rearranging the silicon-protecting group compounds with sulfur dioxide extrusion via thermal isomerization to yield a silicon-protected 5,6-trans-vitamin D₃;
oxidizing 5,6-trans-vitamin D₃ via allylic oxidation to yield a 1α-hydroxy derivative;
de-protecting the 1α-hydroxy derivative to yield crystalline 1α-hydroxy-5,6-trans-vitamin D₃; and
photochemically isomerizing the crystalline 1α-hydroxy-5,6-trans-vitamin D₃ to yield 1α-hydroxy-vitamin D₃, wherein the 1α-hydroxy-vitamin D₃ has a melting point of about 140° C. to about 144° C.

31. The method of claim 30, further comprising the steps of purifying the 1α-hydroxy-vitamin D₃ via polish filtration and recrystallizing the 1α-hydroxy-vitamin D₃ via a solvent exchange with at least one solvent selected from the group consisting of n-heptane, heptanes, and a combination thereof.

32. The method of claim 31, wherein the at least one solvent is n-heptane.

33. The method of claim 30, wherein the 1α-hydroxy-vitamin D₃ is 1α,3β,5Z,7E-9,10-secocholesta-5,7,10(19)-triene-1,3-diol.

34. The method of claim 30, further comprising the steps of drum-drying or spray-drying the 1α-hydroxy-vitamin D₃ and mixing the 1α-hydroxy-vitamin D₃ with a carrier.

35. The method of claim 30, wherein the yield of 1α-hydroxy-5,6-trans-vitamin D₃ is at least about 30%.

36. The method of claim 30, further comprising the steps of dissolving the 1α-hydroxy-5,6-trans-vitamin D₃ in tetrahydrofuran, recovering solid 1α-hydroxy-5,6-trans-vitamin D₃, and treating the 1α-hydroxy-5,6-trans-vitamin D₃ with n-heptane.

37. The method of claim 36, wherein the purity of the 1α-hydroxy-5,6-trans-vitamin D₃ is at least about 94.5%.

38. The method of claim 30, wherein the purity of the 1α-hydroxy-vitamin D₃ is at least 96%.

39. The method of claim 30, wherein the step of photochemically isomerizng the crystalline 1α-hydroxy-5,6-trans-vitamin D₃ is monitored by HPLC.

40. A compound, 1α,3β,5Z,7E-9,10-secocholesta-5,7,10(19)-triene-1,3-diol, produced by the method of claim 30.

41. The compound of claim 40, which has a melting point of about 140° C. to about 144° C.

42. The compound of claim 40, which is characterized by a melt onset at about 141° C.

43. The compound of claim 40, wherein the compound is present substantially as a single polymorph.
